(19) United States
(12) Patent Application Publication
(10) Pub. No.: US 2026/0130797 A1
(43) Pub. Date: May 14, 2026

(54) SHUNTING SYSTEMS HAVING FLOW CONTROL ASSEMBLIES WITH ADJUSTABLE LUMENS OF INTERLACED COMPOSITION AND ASSOCIATED SYSTEMS, METHODS, AND DEVICES

(71) Applicant: Shifamed Holdings, LLC, Campbell, CA (US)

(72) Inventors: Eric Schultz, Los Altos, CA (US); Nicholas Lyford, Sunnyvale, CA (US)

(21) Appl. No.: 18/705,165

(22) PCT Filed: Nov. 3, 2022
(86) PCT No.: PCT/US2022/048863

§ 371 Date: Apr. 26, 2024

Related U.S. Application Data
(60) Provisional application No. 63/280,502, filed on Nov. 17, 2021.

(51) Int. Cl. A61F 9/007

(52) U.S. Cl. CPC A61F 9/00781

(57) ABSTRACT

The present technology is generally directed to adjustable shunting systems, including adjustable shunting systems with flow control assemblies having adjustable lumens. The flow control assembly can include a body that defines a lumen. The body can include an adjustable portion having an inner width or diameter that is inversely proportional to a fluid resistance of the lumen. The flow control assembly can be configured to transition between one or more configurations, and each configuration can correspond to a different inner width of the adjustable portion. The flow control assembly can be translation-based, such that transitioning the flow control assembly between the one or more configurations can include translating at least part of the flow control assembly.

### CROSS-REFERENCE TO RELATED APPLICATION(S)

The present application claims priority to U.S. Provisional Patent Application No. 63/280,502, filed Nov. 17, 2021, the disclosure of which is incorporated by reference herein in its entirety.

### TECHNICAL FIELD

The present technology generally relates to implantable medical devices and, in particular, to adjustable shunting systems and associated methods for selectively controlling fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen(s)). Conventional, early shunting systems (sometimes referred to as minimally invasive glaucoma surgery devices or “MIGS” devices) have shown clinical benefit; however, there is a need for improved shunting systems, systems for delivering such shunting systems, and techniques for addressing elevated intraocular pressure and risks associated with glaucoma. For example, there is a need for shunting systems capable of adjusting the therapy provided to meet the patient's individual and variable needs and/or account for changes in flow-related characteristics, including the flow rate between the two fluidly connected bodies.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1 is a partially schematic illustration of a flow control assembly configured in accordance with various embodiments of the present technology.

FIGS. 2A-2C are partially schematic illustrations of a flow control assembly configured in accordance with various embodiments of the present technology.

FIG. 3 is a partially schematic illustration of a portion of an actuation mechanism for adjusting a flow control assembly and configured in accordance with various embodiments of the present technology.

FIGS. 4A-4D are partially schematic illustrations of a sequence of operations of an actuation mechanism to adjust a fluid resistance of a flow control assembly configured in accordance with various embodiments of the present technology.

### DETAILED DESCRIPTION

The present technology is generally directed to adjustable shunting systems, including adjustable shunting systems with flow control assemblies having adjustable lumens. The flow control assembly can include a body that defines a lumen. The flow control assembly can be integrated into a body that defines a lumen. The body can include an adjustable portion defining an inner dimension (e.g., width, diameter, etc.) of the lumen, having a fluid resistance that is proportional thereto. The flow control assembly can be configured to transition between one or more configurations, and each configuration can correspond to a different inner dimension of the adjustable portion. The flow control assembly can comprise an interlaced (e.g., braided or woven) structure. The flow control assembly can be translation-based, such that transitioning the flow control assembly between the one or more configurations can include translating at least a portion of the flow control assembly with respect to another portion. For example, in response to extension of a first portion with respect to a remainder of the flow control assembly, the flow control assembly can be configured to constrict the adjustable portion inwardly (e.g., toward a longitudinal axis of the body) to reduce the inner dimension of the adjustable portion. Reducing the inner dimension of the adjustable portion can increase the fluid resistance of the flow control assembly. For example, in response to compression of a first portion with respect to a remainder of the flow control assembly, the flow control assembly can be configured to expand the adjustable portion outwardly (e.g., away from a longitudinal axis of the body) to increase the inner dimension of the adjustable portion. Increasing the inner dimension of the adjustable portion can decrease the fluid resistance of the flow control assembly.

As described in greater detail below, it is expected that in at least some embodiments the present technology may exhibit one or more advantageous characteristics that improve shunting systems. For example, shunting systems having flow control assemblies configured in accordance with embodiments of the present technology can include a single lumen defining a single flow path that is adjustable to provide a range of fluid resistances. Accordingly, adjustable shunting systems including flow control assemblies in accordance with embodiments of the present technology are expected to provide a titratable therapy while still being relatively small and relatively easy to implement. Additionally, flow control assemblies configured in accordance with embodiments of the present technology are expected to exhibit a greater degree of adjustability compared to many shunting systems. For example, the translational transitioning of the flow control assemblies described herein can result in incremental or gradual changes to the inner dimension of the adjustable portion. Such incremental changes can allow the flow control assembly to be adjusted between a greater number of different fluid resistances compared to many shunting systems. Of course, the present technology may also provide additional advantageous characteristics not expressly described herein.

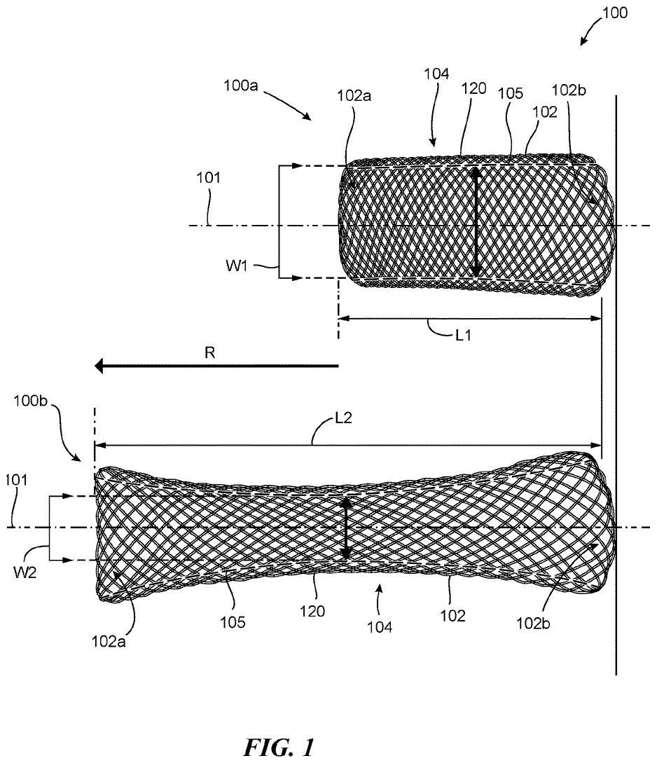

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1-4D.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics described herein may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye (including the posterior chamber), or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

In some embodiments, an adjustable portion includes a structure that comprises a plurality of interlaced elements, for example threads or filaments, that are arranged in a weave or a braid. The braid can be a cylindrical, helically wound braid, for example a biaxial braid. In operation, an applied tension force along the adjustable portion (e.g., a pull to at least one end) serves to lengthen and narrow the adjustable portion. Without being bound by theory, the length is gained by reducing the angle between the (e.g., warp and weft) threads at their crossing points. This reduction in angle is accompanied by a reduction in the radial distance between opposing sides of the adjustable portion, and hence the overall circumference. The more the adjustable portion is pulled (to a limit), the greater the reduction in circumference and the inner dimension (e.g., lumen). Conversely, an applied compression force to the adjustable portion (e.g., a push to at least one end) serves to shorten and widen the adjustable portion (and the lumen thereof).

FIG. 1 is a partially schematic illustration of a flow control assembly 100 (“assembly 100”) configured to have at least two states/configurations (shown in FIG. 1, for example, as a first state 100a and a second state 100b) in accordance with various embodiments of the present disclosure. As described in greater detail below, the assembly 100 can comprise and/or be incorporated into an adjustable shunting system and used to selectively adjust or titrate the fluid resistance through the shunting system. The assembly 100 can be incorporated into any suitable shunting system having a lumen, such as any of the adjustable shunting systems incorporated by reference herein, and/or any other suitable adjustable shunting system.

The assembly 100 can include a generally elongate body 102 (which can also be referred to as a shunting element, a conduit, or the like) having a first end portion 102a, a second end portion 102b opposite the first end portion 102a, and an adjustable portion 104 extending between the first end portion 102a and the second end portion 102b. The adjustable portion 104 can define an inner contour or shape of the body 102 having one or more dimensions (e.g., width, diameter, etc.) of the body 102. The assembly 100 can be partially or fully hollow, such that the body 102 can define a lumen 105 through which fluid (e.g., aqueous) can flow. In the illustrated embodiment, the body 102 has a generally circular cross-sectional shape. In other embodiments, however, the body 102 can have an oval, triangular, square, rectangular, pentagonal, hexagonal, rectilinear, curvilinear, and/or any other suitable cross-sectional shape.

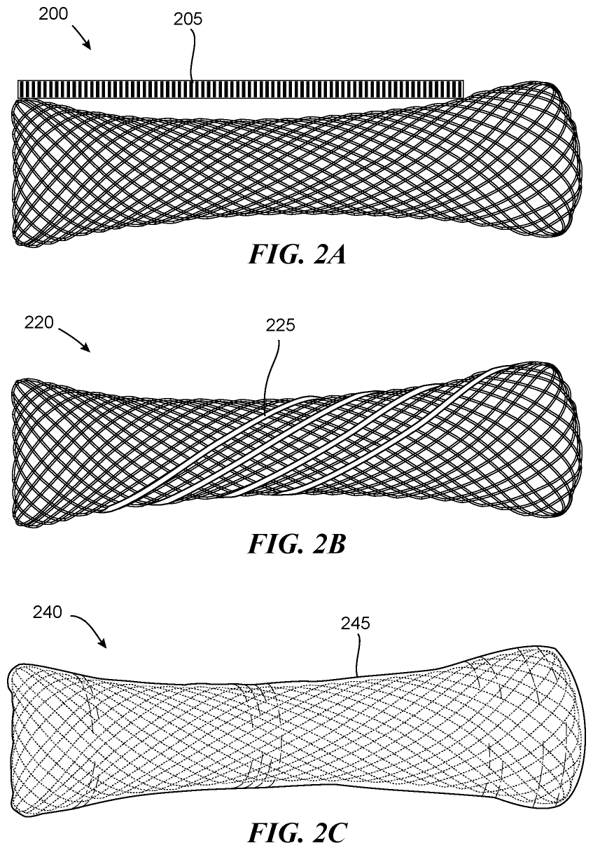

The adjustable portion 104 in FIG. 1 can include a plurality of interlaced elements 120 (e.g., spines, filaments, fibers, or threads) that are interwoven with respect to each other. The interlaced elements 120 can be covered by a flexible and impermeable membrane (see, for example, FIG. 2C) or the like such that the fluid that flows through the adjustable portion 104 is generally or substantially prevented from leaking from the assembly 100 (e.g., at least at locations along the adjustable portion 104). The interlaced elements 120 can at least partially define a contour, profile, and/or minimum dimension of a portion (e.g., the adjustable portion 104) of the body. In at least some embodiments, for example, the interlaced elements 120 can define a longitudinal or lengthwise contour or shape (e.g., cylindrical, hourglass-shaped, etc.) of the adjustable portion 104 relative to a longitudinal axis 101 of the body, e.g., between the longitudinal axis 101 and individual ones of the interlaced elements 120.

As noted previously, in operation, the assembly 100 can be transitioned between one or more states/configurations. In the illustrated embodiment, for example, the assembly 100 can be transitionable between: (i) the first state/configuration 100a in which the lumen 105 of the adjustable portion 104 has a first inner dimension (e.g., a first width W1, a first inner diameter, etc.) and a first extent (e.g., a first length L1); and (ii) the second state/configuration 100b in which the lumen 105 of the adjustable portion 104 has a second inner dimension (e.g., a second width W2, a second inner diameter, etc.) less than the first width W1, and a second extent (e.g., a second length L2) greater than the first extent. The inner dimensions (e.g., inner widths W1, W2) of the lumen 105 within the adjustable portion 104 (e.g., the dimensions of the inner contour defined by the adjustable portion 104) can correspond to (e.g., be inversely proportional to, or at least partially confer) a fluid resistance of the assembly 100. For example, the assembly 100 can provide: (i) a first resistance when in the first configuration 100a having dimensions W1 and L1, and (ii) a second resistance greater than the first resistance when in the second configuration 110b having dimensions W2 and L2.

In some embodiments, the adjustable portion 104 is incrementally adjustable (e.g., in discrete steps). In some embodiments, the adjustable portion 104 is continuously adjustable. While depicted in FIG. 1 as two states/configurations, it will be appreciated that the assembly 100 can assume a plurality of states/configurations having dimensions that range from width W1 to width W2, and from length L1 to length L2. The assembly 100 can include at least 3, 5, 6, or any other suitable number of states/configurations. Thus, for a given fluid pressure, the various resistances provided by the various states/configurations can correspond to a flow rate at which the fluid can flow through the assembly 100 (e.g., to drain from a first body region to a second, different body region of a patient, such as to drain aqueous from an anterior chamber of the patient's eye). For example, under a given pressure, the assembly 100 can provide: (i) a first flow rate when in the first state/configuration 100a, and (ii) a second flow rate less than the first flow rate when in the second state/configuration 100b. Accordingly, the relative level of therapy provided by each of the configurations can be different so that a user may adjust the level of therapy provided by the assembly 100 by selectively increasing and/or decreasing the inner width of the adjustable portion 104.

The assembly 100 can be transitioned between one or more of the states/configurations by manipulating one or more portions of the assembly 100. For example, the assembly 100 can be transitioned between the states/configurations by extending or compressing the first end portion 102a of the body 102 with respect to the second end portion 102b of the body 102. The second end portion 102b can remain fixed with respect to a remainder of the assembly 100 and/or to a fixed position within the body of a patient in which the assembly 100 is implanted (e.g., via a suture). As illustrated in FIG. 1, translating the first end portion 102a relative to the second end portion 102b in the direction R can cause the interlaced elements 120 (and/or any membrane coupled thereto) to reduce an angle between the (e.g., warp and weft) elements at their crossing points. This reduction in angle is accompanied by a reduction in the radial distance between opposing sides of the adjustable portion 104, and hence the overall circumference. Transitioning the assembly 100 between one or more of the states/configurations can cause a corresponding change in a contour or shape of the adjustable portion 104. For example, transitioning the assembly 100 from the first state/configuration 100a to the second state/configuration 100b can change the shape of the adjustable portion 104 from a generally cylindrical shape to a generally hourglass shape, such that the adjustable portion 104 can constrict or taper inwardly toward the longitudinal axis 101 of the body 102 at or near the center or central region of the adjustable portion 104. In some embodiments, the assembly 100 can be configured to operate in reverse, for example, by translating the first end portion 102a toward the second end portion 102b, and thereby increasing the inner dimension of the lumen 105 and reducing fluid resistance through the assembly 100.

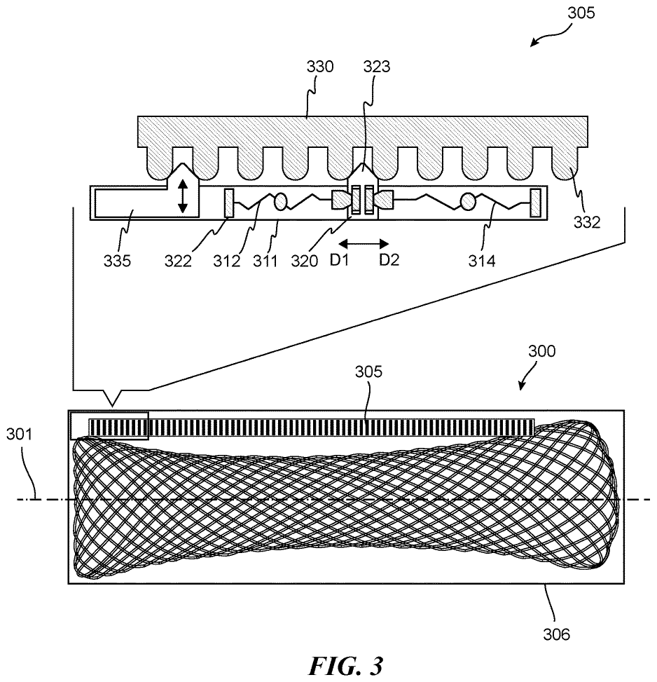

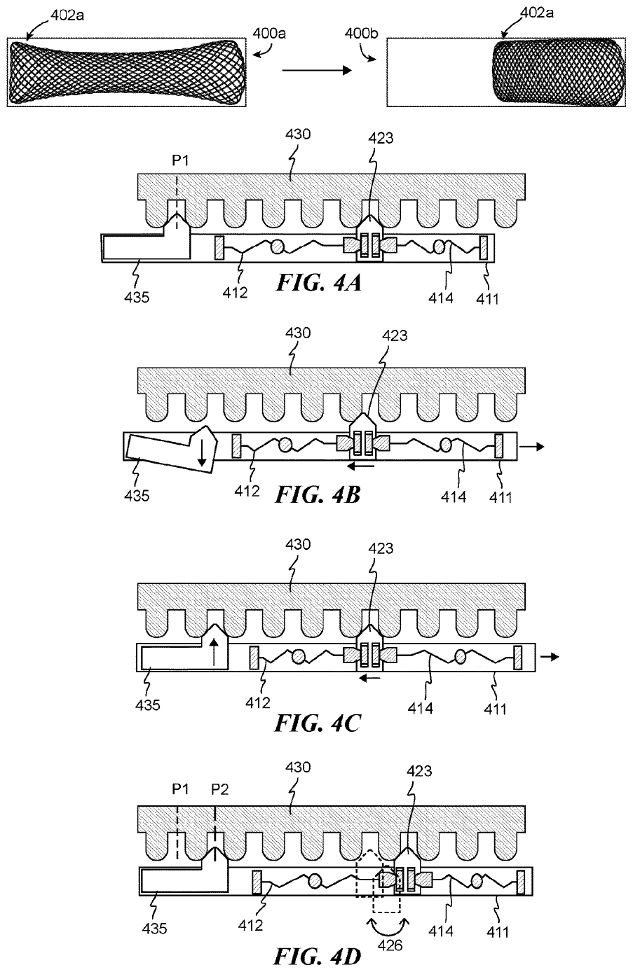

FIGS. 2A-2C illustrate various embodiments of a flow control assembly according to the present disclosure. FIG. 2A depicts a flow control assembly 200 having an actuation mechanism 205 coupled thereto. Actuation mechanism 205 is operable to (e.g., reversibly) transition the actuation assembly 200 at least from a first state/configuration (e.g., 100a of FIG. 1) to a second state/configuration (e.g., 100b of FIG. 1). The operation of actuation mechanism 205 is described in greater detail in FIG. 3 and in FIGS. 4A-4D.

FIG. 2B illustrates a flow control assembly 220 having embedded shape memory elements 225. The shape memory elements 225 can be formed to a have a selected memory geometry (e.g., shape set), which geometry can be recovered by application of energy following a deformation therefrom. The shape memory elements 225 can be generally longitudinally arranged. The geometry of the shape memory elements 225 can be generally helical, coiled, or spiraled. Examples of applied energy include electromagnetic (e.g., laser, radiofrequency (RF)) energy, and thermal energy. In some embodiments, the shape memory elements 225 can be formed to have an elongated shape (e.g., shape set corresponding to configuration 100b of FIG. 1). In operation, when the assembly 220 is in a compressed state (e.g., configuration 100a of FIG. 1), energy applied to the shape memory elements 225 can cause recovery to the shape memory geometry and transition the assembly 220 to an elongated state (e.g., configuration 100b). In some embodiments, shape memory elements 225 can be formed to have a compressed shape (e.g., shape set corresponding to configuration 100a), and application of energy to shape memory elements 225 deformed by elongation can urge the assembly 220 toward its compressed state (e.g., configuration 100a). In some embodiments, shape memory elements 225 comprise a mixture of shape memory geometries, where given elements have varied shape set lengths/geometries. The shape memory elements 225 can be formed of an alloy comprising nickel and titanium composite (e.g., Nitinol), or from a polymer (e.g., shape memory polymer, SMP). The shape memory elements can be biocompatible.

FIG. 2C illustrates a flow control assembly 240 comprising a coating or covering 245. The coating can comprise, for example, silicone, polymethylmethacrylate (PMMA), or polydimethylsiloxane (PDMS). Actuation elements (e.g., 205 (FIGS. 2A) and/or 225 (FIG. 2B)) can be embedded within, carried on, and/or covered by the coating 245. In some embodiments, the coating 245 is impregnated with or otherwise includes a radioactive material that emits (e.g., alpha or beta) radiation. In some embodiments, the coating 245 can encourage tissue growth (e.g., infiltration). In some embodiments, the coating 245 can inhibit tissue growth. The coating 245 can be substantially fluid impermeable. The coating 245 can be applied to at least a portion (e.g., an entirety) of the assembly 240.

Turning now to FIG. 3, an embodiment of an actuation mechanism 305 that operable to adjust a configuration of a flow control assembly 300 is depicted. The actuation mechanism 305 can be at least a portion of a flow control assembly, such as the assembly 100 of FIG. 1, the assembly 200 of FIG. 2A, or any other suitable flow control assembly, and can be used to selectively drive the assembly incrementally through various configurations to adjust the fluid resistance provided by the respective flow control assembly.

The actuation mechanism 305 can include a drive element 330, a pawl or contact element 323, and one or more actuation elements (shown as a first actuation element 312 and a second actuation element 314 in FIG. 3). The actuation element(s) 312 and 314 and the pawl 323 can be operably coupled to the drive element 330. The drive element 330 can be coupled to the body of the assembly (e.g., to the first end portion 102a) such that, as described in greater detail below, the actuation element(s) 312, 314 and the pawl 323 can be operable to translate the drive element 330 and the first end portion 102a, thereby transitioning the assembly between one or more of the states/configurations (FIGS. 1, 100a and 100b). The drive element 330 can include one or more engagement features 332 positioned along an edge of the drive element 330. Each of the engagement features 332 can include generally symmetrical surfaces (e.g., functioning as a drive surface and a return surface). The drive element 330 can include a suitable number of engagement features 332 to transition the assembly from at least the first configuration to the second configuration. In at least some embodiments, the number of engagement features 332 can correspond to the number of configurations of the assembly 300. As described in greater detail below, each of the engagement features 332 can correspond to a translational increment of a configuration of a flow control assembly 300.

For simplicity of illustration, like reference numbers for actuation element 314 are omitted, although the components and manner of operation is similar as that described for actuation element 312. Each of the actuation elements (e.g., 312 and 314) can include a first end portion 320 coupled to the pawl 323. Each of the actuation elements 312 and 314 can be actuated to move the pawl 323 to contact at least a portion of one of the engagement features 332 of the drive element 330. A locking element 335 may (e.g., selectively) engage with engagement features 332 to at least temporarily fix a position of the drive element 330 with respect to the adjustable assembly 300, for example, in between actuations of the first or second actuation elements 312 and 314. Each of the actuation elements can include corresponding second end portions 322 that, along with the locking element 335, can have fixed positions relative to an axis 301 and/or a support body 311 of the assembly 300. In some embodiments, the drive element 330 is (e.g., fixedly) coupled to a surface of a housing 306 that carries or otherwise encapsulates the adjustable assembly 300, while the support body 311 is (e.g., fixedly) coupled to a portion of the adjustable assembly 300 (e.g., end 102a of FIG. 1). As described in greater detail below with reference to FIGS. 4A-4D, actuating the actuation elements 312 or 314 can drive translation (e.g., incremental translation) of the drive element 330.

Each of the actuation elements 312 and 314 can be composed at least partially of a shape memory material or alloy (e.g., Nitinol). Accordingly, each actuation elements 312 and 314 can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the actuation elements 312 and 314 may have reduced (e.g., relatively less stiff) mechanical properties that cause the actuation elements to be more easily deformable (e.g., compressible, expandable, etc.) relative to when the actuation elements are in the first material state. In the second material state, the actuation elements 312 and 314 may have increased (e.g., relatively stiffer) mechanical properties relative to the first material state, causing an increased preference toward a specific preferred geometry (e.g., original geometry, manufactured or fabricated geometry, heat set geometry, etc.). In the illustrated embodiment, the first actuation element 312 and the second actuation element 314 can be selectively and independently transitioned between the first material state and the second material state by applying energy (e.g., laser energy, electrical energy, etc.) to the first actuation element 312 or the second actuation element 314 to heat it above a transition temperature (e.g., above an austenite finish (A_{f}) temperature, which is generally greater than body temperature). If the first actuation element 312 (or the second actuation element 314) is deformed relative to its preferred geometry when heated above the transition temperature, the first actuation element 312 (or the second actuation element 314) will move to and/or toward its preferred geometry. In some embodiments, the first actuation element 312 and the second actuation element 314 are operably coupled such that, when the actuated actuation element (e.g., the first actuation element 312) transitions toward its preferred geometry, the non-actuated actuation element (e.g., the second actuation element 314) is further deformed relative to its preferred geometry.

The first actuation element 312 and the second actuation element 314 generally act in opposition. During operation, for example, the first actuation element 312 can be actuated to move the pawl 323 in a first direction D1, and the second actuation element 314 can be actuated to move the pawl 323 in a second direction D2 generally or substantially opposite the first direction D1. Additionally, as described above, the first actuation element 312 and the second actuation element 314 can be coupled such that, as one moves toward its preferred geometry upon material phase transition, the other is deformed relative to its preferred geometry. This enables the actuation elements 312 and 314 to be repeatedly actuated and the pawl 323 to be repeatedly cycled in the first direction DI and/or the second direction D2. Additional details regarding the operation of shape memory actuators for use with adjustable shunts are described in International Patent Application No. PCT/US21/27742, filed Apr. 16, 2021, the disclosure of which is incorporated by reference herein in its entirety and for all purposes.

In operation, each of the actuation elements can be actuated to drive movement of the pawl relative to the support body, e.g., to drive translation of the adjustable assembly via the support body that is coupled to an end thereof. Referring now to FIGS. 4A-4D, an incremental movement of an adjustable assembly from a first state/configuration 400a toward a second state/configuration 400b is illustrated. To aid in description, the adjustable assembly body is omitted from FIGS. 4A-4D, and only a portion of a drive element 430, along with actuation elements 412 and 414 and a locking 435 element 435 carried by a support body 411 are depicted. In this description, it will be appreciated that movement of support body 411 is also reflective of movement of the portion of an adjustable assembly (e.g., end 402a) to which the support body is coupled.

Beginning with FIG. 4A, the support body 411 is at an initial position P1 with respect to the drive element 430 (as referenced by a position of locking element 435). At FIG. 4B, actuation of actuation element 412 causes a contraction thereof, pulling pawl 423 against the drive element 430. At a same time, the locking element 435 is disengaged from the drive element 430. The force of the pawl 423 against the drive element 430 is transmitted through the actuation element 412 to the support body 411, causing the support body 411 to advance in an opposing direction. The actuation element 414, which remains in a non-actuated state (e.g., soft, or martensitic), is elongated. At FIG. 4C the contraction of actuation element 412 continues, further pulling the pawl 423 against the drive element 430, advancing the support body 411, and elongating actuation element 414. At the completion of the actuated movement of actuation element 412, locking element 435 is returned to a locking position to maintain the incremented position of the support body 411. At FIG. 4D, a recoil of the actuation element 412 occurs, which, along with an excursion 426 of pawl 423, resets the position of the actuation elements 412 and 414 along with that of the pawl 423 while maintaining the support body 411 at new position P2. Coupled actuation elements that utilize recoil to selectively drive movement in opposing directions are described in International Patent Application No. PCT/US21/27742, which was previously incorporated herein by reference.

The actuation steps as described in FIGS. 4A-4D can be repeated in order to incrementally advance one end of the adjustable assembly toward another, for example to move the adjustable assembly from configuration 400a toward configuration 400b and thereby reduce a fluid resistance therethrough. The adjustable assembly of the present disclosure can operate in the reverse, where actuator 414 is actuated to incrementally move an end of the adjustable assembly away from an opposing end, thereby increasing a fluid resistance (e.g., from 400b toward 400a). While the actuation of the example is a contraction, it will be appreciated that in alternative embodiments actuators are extended during actuation.

As one skilled in the art will appreciate, one or more of the flow control assemblies and/or actuation mechanisms described above can be used as part of an adjustable shunting system, e.g., to control the flow of fluid therethrough. Moreover, certain features described with respect to one flow control assembly or actuation mechanism can be added or combined with another flow control assembly or actuation mechanism. Accordingly, the present technology is not limited to the flow control assemblies and actuation mechanisms expressly identified herein.

### EXAMPLES

Several aspects of the present technology are set forth in the following examples:
1. A flow control assembly for use with a shunting system for shunting fluid between a first body region and a second body region, the flow control assembly comprising:
a hollow body having a first end portion, a second end portion opposite the first end portion, and an adjustable portion extending between the first end portion and the second end portion, the adjustable portion comprising an inner dimension having an associated fluid resistance of the flow control assembly; and
an actuation mechanism operably coupled to the hollow body, wherein the actuation mechanism is configured to translate the first end portion relative to the second end portion to selectively change the inner dimension of the adjustable portion of the hollow body.
2. The flow control assembly of example 1 wherein, when actuated, the actuation mechanism is configured to translate the first end portion toward the second end portion to increase the inner dimension.
3. The flow control assembly of example 1 wherein, when actuated, the actuation mechanism is configured to translate the first end portion away from the second end portion to decrease the inner dimension.
4. The flow control assembly of any of examples 1-3, further comprising a housing, wherein at least a portion of the body is positioned within the housing.
5. The flow control assembly of example 4 wherein at least a portion of the actuation mechanism is operably coupled to the housing and configured to translate relative to the housing.
6. The flow control assembly of example 5 wherein the first end portion of the body and the housing are configured such that the actuation mechanism is operable to translate the first end portion of the body between one or more increments, and wherein each increment corresponds to a different inner dimension of the adjustable portion.
7. The flow control assembly of example 5 wherein the actuation mechanism further comprises:
an actuation element;
a pawl; and
a drive element comprising a plurality of engagement features,
wherein the actuation mechanism is configured to force the pawl against a first engagement feature to cause the first end portion of the hollow body to translate, and subsequently to advance the pawl to a second engagement feature.
8. The flow control assembly of any of examples 1-7 wherein the adjustable portion comprises a plurality of interlaced elements.
9. The flow control assembly of example 8 wherein the interlaced elements comprise a braid or weave.
10. The flow control assembly of example 8 or example 9 wherein the actuation mechanism is at least partially integrated into a wall of the hollow body.
11. The flow control assembly of example 10 wherein the actuation mechanism includes a plurality of longitudinal shape memory elements extending at least partially between the first end portion and the second end portion.
12. The flow control assembly of any of examples 1-11 wherein the actuation mechanism comprises a shape memory metal or shape memory polymer.
13. A method for selectively controlling fluid flow from a first body region to a second body region through a shunting system having a flow control assembly, the method comprising:
applying energy to an actuation element of an actuation mechanism of the flow control assembly; and
in response to the applied energy, translating a support body of the actuation mechanism that is coupled with the adjustable portion,
wherein translating the support body includes translating a first end of the adjustable portion relative to a second end of the adjustable portion to change an inner dimension of an adjustable portion of the flow control assembly.
14. The method of example 13 wherein translating the support body includes transitioning the adjustable portion between a first configuration in which the adjustable portion has a first inner dimension and a second configuration in which the adjustable portion has a second inner dimension less than the first inner dimension.
15. The method of example 13 wherein translating the first end toward the second end comprises increasing the inner dimension, and further wherein translating the first end away from the second end comprises reducing the inner dimension.
16. The method of any of examples 13-15 wherein applying energy to the actuation element includes forcing a pawl of the actuation mechanism in a first direction to drive movement of the support body in an opposing direction.
17. The method of example 16 wherein the actuation element is a first actuation element, and wherein the method further comprises:
applying energy to a second actuation element of the actuation mechanism; and
in response to the applied energy, forcing the pawl in a second direction opposite the first direction to drive movement the support body in the first direction.
18. The method of any of examples 13-17 wherein translating the first end of the adjustable portion relative to the second end of the adjustable portion comprises transitioning at least a portion of one or more longitudinal elements of the flow control assembly from a first geometry toward a second geometry, relative to a longitudinal axis of the adjustable portion, the one or more longitudinal elements extending at least partially between the first end and the second end.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the adjustable shunts described herein may be combined with any of the features of the other adjustable shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A flow control assembly for use with a shunting system for shunting fluid between a first body region and a second body region, the flow control assembly comprising:
a hollow body having a first end portion, a second end portion opposite the first end portion, and an adjustable portion extending between the first end portion and the second end portion, the adjustable portion comprising an inner dimension having an associated fluid resistance of the flow control assembly; and
an actuation mechanism operably coupled to the hollow body, wherein the actuation mechanism is configured to translate the first end portion relative to the second end portion to selectively change the inner dimension of the adjustable portion of the hollow body.

2. The flow control assembly of claim 1 wherein, when actuated, the actuation mechanism is configured to translate the first end portion toward the second end portion to increase the inner dimension.

3. The flow control assembly of claim 1 wherein, when actuated, the actuation mechanism is configured to translate the first end portion away from the second end portion to decrease the inner dimension.

4. The flow control assembly of claim 1, further comprising a housing, wherein at least a portion of the body is positioned within the housing.

5. The flow control assembly of claim 4 wherein at least a portion of the actuation mechanism is operably coupled to the housing and configured to translate relative to the housing.

6. The flow control assembly of claim 5 wherein the first end portion of the body and the housing are configured such that the actuation mechanism is operable to translate the first end portion of the body between one or more increments, and wherein each increment corresponds to a different inner dimension of the adjustable portion.

7. The flow control assembly of claim 5 wherein the actuation mechanism further comprises:
an actuation element;
a pawl; and
a drive element comprising a plurality of engagement features,
wherein the actuation mechanism is configured to force the pawl against a first engagement feature to cause the first end portion of the hollow body to translate, and subsequently to advance the pawl to a second engagement feature.

8. The flow control assembly of claim 1 wherein the adjustable portion comprises a plurality of interlaced elements.

9. The flow control assembly of claim 8 wherein the interlaced elements comprise a braid or weave.

10. The flow control assembly of claim 8 wherein the actuation mechanism is at least partially integrated into a wall of the hollow body.

11. The flow control assembly of claim 10 wherein the actuation mechanism includes a plurality of longitudinal shape memory elements extending at least partially between the first end portion and the second end portion.

12. The flow control assembly of claim 1 wherein the actuation mechanism comprises a shape memory metal or shape memory polymer.

13. A method for selectively controlling fluid flow from a first body region to a second body region through a shunting system having a flow control assembly, the method comprising:
applying energy to an actuation element of an actuation mechanism of the flow control assembly; and
in response to the applied energy, translating a support body of the actuation mechanism that is coupled with the adjustable portion,
wherein translating the support body includes translating a first end of the adjustable portion relative to a second end of the adjustable portion to change an inner dimension of an adjustable portion of the flow control assembly.

14. The method of claim 13 wherein translating the support body includes transitioning the adjustable portion between a first configuration in which the adjustable portion has a first inner dimension and a second configuration in which the adjustable portion has a second inner dimension less than the first inner dimension.

15. The method of claim 13 wherein translating the first end toward the second end comprises increasing the inner dimension, and further wherein translating the first end away from the second end comprises reducing the inner dimension.

16. The method of claim 13 wherein applying energy to the actuation element includes forcing a pawl of the actuation mechanism in a first direction to drive movement of the support body in an opposing direction.

17. The method of claim 16 wherein the actuation element is a first actuation element, and wherein the method further comprises:
applying energy to a second actuation element of the actuation mechanism; and
in response to the applied energy, forcing the pawl in a second direction opposite the first direction to drive movement the support body in the first direction.

18. The method of claim 13 wherein translating the first end of the adjustable portion relative to the second end of the adjustable portion comprises transitioning at least a portion of one or more longitudinal elements of the flow control assembly from a first geometry toward a second geometry, relative to a longitudinal axis of the adjustable portion, the one or more longitudinal elements extending at least partially between the first end and the second end.
